(19) United States
(12) Patent Application Publication
(10) Pub. No.: US 2021/0353218 A1
(43) Pub. Date: Nov. 18, 2021

(54) Machine Learning Systems and Methods for Multiscale Alzheimer's Dementia Recognition Through Spontaneous Speech

(71) Applicant: Insurance Services Office, Inc., Jersey City, NJ (US)

(72) Inventors: Erik Edwards, Oakland, CA (US); Charles Dognin, Long Beach, NY (US); Bajibabu Bollepalli, Narasaraopet (IN); Maneesh Kumar Singh, Princeton, NJ (US)

(73) Assignee: Insurance Services Office, Inc., Jersey City, NJ (US)

(21) Appl. No.: 17/322,047

(22) Filed: May 17, 2021

Related U.S. Application Data
(60) Provisional application No. 63/026,032, filed on May 16, 2020.

(51) Int. Cl. A61B 5/00; G10L 15/02; G10L 25/66; G10L 15/14; G10L 15/22; G16H 50/20

(52) U.S. Cl. CPC A61B 5/4803; G10L 15/02; G10L 25/66; G10L 15/14; G10L 2015/025; A61B 5/4088; A61B 5/7267; G16H 50/20; G10L 15/22

(57) ABSTRACT

Machine learning systems and methods for multiscale Alzheimer's dementia recognition through spontaneous speech are provided. The system retrieves one or more audio samples and processes the one or more audio samples to extract acoustic features from audio samples. The system further processes the one or more audio samples to extract linguistic features from the audio samples. Machine learning is performed on the extracted acoustic and linguistic features, and the system indicates a likelihood of Alzheimer's disease based on output of machine learning performed on the extracted acoustic and linguistic features.

### RELATED APPLICATIONS

This application claims priority to U.S. Provisional Patent Application Ser. No. 63/026,032 filed on May 16, 2020, the entire disclosure of which is hereby expressly incorporated by reference.

### BACKGROUND

### Technical Field

The present disclosure relates generally to machine learning systems and methods. More specifically, the present disclosure relates to machine learning systems and methods for multiscale Alzheimer's dementia recognition through spontaneous speech.

### Related Art

Alzheimer's disease (AD) is the most common cause of dementia, a group of symptoms affecting memory, thinking and social abilities. Detecting and treating the disease early is important to avoid irreversible brain damage. Several machine-learning (ML) approaches to identify probable AD and MCI (Mild Cognitive Impairment) have been developed in an effort to automate and scale diagnosis.

Detection of AD using only audio data could provide a lightweight and non-invasive screening tool that does not require expensive infrastructure, and can be used in peoples' homes. Speech production with AD differs qualitatively from normal aging or other pathologies, and such differences can be used for early diagnosis of AD. Several studies have been proposed to detect AD using speech signals, and have shown that spectrographic analysis of temporal and acoustic features from speech can characterize AD with high accuracy. Other studies have used only acoustic features extracted from the recordings of DementiaBank for AD detection, and reported accuracy results of up to 97%.

There has also been recent work in text-based diagnostic classification approaches. These techniques use either engineered features or deep features. One engineered features approach showed that classifiers trained on automatic semantic and syntactic features from speech transcripts were able to discriminate between semantic dementia, progressive nonfluent aphasia, and healthy controls. This work was later extended to AD vs healthy control classification using lexical and n-gram linguistic biomarkers.

Deep learning models to automatically detect AD have also recently been proposed. One such system introduced a combination of deep language models and deep neural networks to predict MCI and AD. One limitation of a deep-learning-based approach is the paucity of training data typical in medical settings. Another study has attempted to interpret what the neural models learned about the linguistic characteristics of AD patients. Text embeddings of transcribed text have also been recently explored for this task. For instance, Word2Vec and GloVe have been successfully used to discriminate between healthy and probable AD subjects, while more recently, multi-lingual FastText embedding combined with a linear SVM classifier has been applied to classification of MCI versus healthy controls.

Multimodal approaches using representations from images have been recently used to detect AD. One such approach used lexicosyntactic, acoustic and semantic features extracted from spontaneous speech samples to predict clinical MMSE scores (indicator of the severity of cognitive decline associated with dementia). Others extended this approach to classification, and obtained state-of-the-art results on DemantiaBank-fused linguistic and acoustic features extracted into a logistic regression classifier. Multimodal and multiscale Deep Learning Approaches to AD detection have also been applied using medical imaging data.

In view of the foregoing, what would be desirable are machine learning systems and methods for multiscale Alzheimer's dementia recognition through spontaneous speech.

### SUMMARY

The present disclosure relates to machine learning systems and methods for multiscale Alzheimer's dementia recognition through spontaneous speech. The system retrieves one or more audio samples and processes the one or more audio samples to extract acoustic features from audio samples. The system further processes the one or more audio samples to extract linguistic features from the audio samples. Machine learning is performed on the extracted acoustic and linguistic features, and the system indicates a likelihood of Alzheimer's disease based on output of machine learning performed on the extracted acoustic and linguistic features.

### BRIEF DESCRIPTION OF THE DRAWINGS

The foregoing features of the invention will be apparent from the following Detailed Description, taken in connection with the accompanying drawings, in which:

FIG. 1 is flowchart illustrating processing steps carried out by the machine learning systems and methods of the present disclosure;

FIGS. 2-3 are charts illustrating testing of the machine learning systems and methods of the present disclosure; and

FIG. 4 is diagram illustrating hardware and software components capable of being utilized to implement the machine learning systems and methods of the present disclosure.

### DETAILED DESCRIPTION

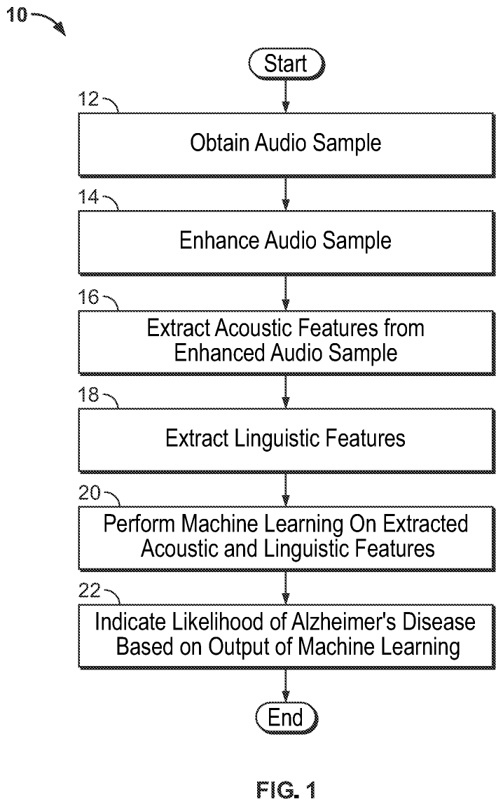

The present disclosure relates to machine learning systems and methods for multiscale Alzheimer's dementia recognition through spontaneous speech, as described in detail below in connection with FIGS. 1-4.

FIG. 1 is a flowchart illustrating processing steps carried out by the machine learning systems and methods of the present disclosure. Beginning in step 12, the system obtains one or more audio samples of individuals speaking particular phrases. Such audio samples could comprise a suitable dataset, such as the dataset provided by the ADReSS Challenge or any other suitable dataset. To generate this dataset of audio samples, the participants were asked to describe the Cookie Theft picture from the Boston Diagnostic Aphasia Exam. Both the speech and corresponding text transcripts were provided. It was released in two parts: train and test sets. The train data had 108 subjects (48 male, 60 female) and the test data had 48 subjects (22 male, 26 female). For the train data, 54 subjects were labeled with AD and 54 with non-AD. The speech transcriptions were provided in CHAT format, with 2169 utterances in the train data (1115 AD, 1054 non-AD), and 934 in the test data.

In step 14, the audio samples are processed by the system to enhance the samples. All audio could be started as 16-bit WAV files at 44.1 kHz sample rate. The audio samples could be provided as ‘chunks,’ which are sub-segments of the above speech dialog segments that have been cropped to 10 seconds or shorter duration (2834 chunks: 1476 AD, 1358 non-AD). The system applies a basic speech-enhancement technique using VOICEBOX, which slightly improved the audio results, but it is noted that this step is optional. Noisy chunks can be rejected, and optionally, a 3-category classification scheme can be used to separately identify the noisiest chunks. Voice activity detection could also be performed, using OpenSMILE or rVAD or any other suitable voice activity detection application, and weighting audio results accordingly. Other methodologies could be utilized to handle the noise levels (e.g., a windowing into fixed-length frames).

In step 16, the system extracts acoustic features from the enhanced audio samples. Acoustic features could be extracted from the enhanced speech segments and downsampled to a 16-kHz sample rate. Then, features are computed every 10-ms to give “low-level descriptors” (LLDs) and then statistical functionals of the LLDs (such as mean, standard deviation, kurtosis, etc.) are computed over each audio chunk of 0.5-10 sec duration (chunks shorter than 0.5 s were rejected). The system extracts the following sets of functionals: emobase, emobase2010, GeMAPS, extended GeMAPS (eGeMAPS), and Com-ParE2016 (a minor update of numerical fixes to the Com-ParE2013 set). The system then extracts multi-resolution cochleagram (MRCG) LLDs, and then several statistical functionals of these LLDs. The dimensions of each functionals set are given in Table 1, below.

As the dimensionality of each functionals set can be large (see Table 1), the system implements feature selection techniques to improve sub-sequent classification. First, the system uses correlation feature selection (CFS), which discards highly-correlated features. Second, the system uses recursive feature elimination with cross validation (RFECV), where a classifier is employed to evaluate the importance of the each feature dimension. In each recursion, the feature that least improves or most degrades classifier importance is discarded, leading to a supervised ranking of features.

Table 1 shows the raw (“All”) feature dimensions and after each feature selection method. Age and gender are appended to each acoustic feature set. With CFS, the system discards features with correlation coefficient ≥0.85. For RFECV, the system uses logistic regression (LR) as the base classifier with leave-one-subject-out (LOSO) cross validation. CFS reduced the dimensionality by 15-95%, and the RFECV method further brought the dimensionality down to 3-54 for all sets.

TABLE 1
Acoustic features and their dimensions. CFS denotes
correlation feature selection and RFECV denotes
recursive feature elimination using cross-validation.

| Feature | Dim. (All) | Dim. (CFS) | Dim. (RFECV) |
|---|---|---|---|
| GEMAPS | 64 | 53 | 3 |
| eGEMAPS | 90 | 76 | 4 |
| emobase | 979 | 626 | 6 |
| emobase2010 | 1583 | 995 | 19 |
| emolarge | 6511 | 1810 | 21 |
| ComParE2016 | 6375 | 3592 | 54 |
| MRCG | 6914 | 367 | 5 |

Table 2, below shows the performance of feature selection methods employed by the system, assessed with LOSO cross-validation on the train set. There is considerable improvement in accuracy after the CFS and RFECV methods. Since the performance of the ComParE2016 features is best among the acoustic feature sets, the system uses the ComParE2016 features for further experiments. However, it is noted that equivalent performance could be obtained with emobase2010 using other feature selection methodology.

TABLE 2
Accuracy scores of feature selection. These numbers
calculated by taking majority vote on segments.

| Feature | (All) | (CFS) | (RFECV) |
|---|---|---|---|
| GEMAPS | 0.490 | 0.472 | 0.629 |
| eGEMAPS | 0.453 | 0.462 | 0.620 |
| emobase | 0.555 | 0.555 | 0.657 |
| emobase2010 | 0.555 | 0.574 | 0.601 |
| emolarge | 0.595 | 0.629 | 0.666 |
| ComParE2016 | 0.601 | 0.629 | 0.694 |
| MRCG | 0.546 | 0.509 | 0.611 |

Table 3, below, presents the accuracy scores achieved by the Com-ParE2016 features using different ML classification models. SVM (support vector machine) and LDA (linear discriminant analysis) models gave better performance than LR. The best accuracy obtained using acoustic features alone is 0.74. The system uses the posterior probabilities from the LDA model averaged over all chunks for each subject.

TABLE 3
Accuracy scores of the ComParE2016 acoustic feature
set with different classifiers.

| Feature | LR | SVM | LDA |
|---|---|---|---|
| ComPareE2016 | 0.694 | 0.740 | 0.740 |

LR: Logistic regression, SVM: sup-port vector machine, and LDA: linear discriminant analysis.

In step 18, the system extracts linguistic features. In this step, two processes are carried out: natural language representation and phoneme representation. For natural language representation, the system applies a basic text normalization to the transcriptions by removing punctuation and CHAT symbols and lower casing. Table 4, below, shows the accuracy and F 1 score results on a 6-fold cross validation of the training data-set (segment level). For each model used, hyper-parameter optimization was performed to allow for fair comparisons.

TABLE 4
Best performance after hyper-parameters optimization for
each model, metrics are the average of accuracy and
f1 scores across 6-fold cross-validation, participant level
(soft max average).

| Model | Dim. | Accuracy | F1-score |
|---|---|---|---|
| Random (DRF) | 11 | 0.463 | 0.482 |
| Engineered Feat (DRF) | 11 | 0.704 | 0.68 |
| Sent2Vec (FT) | 600 | 0.787 | 0.758 |
| GloVe (FT) | 300 | 0.861 | 0.865 |
| Word2Vec (FT) | 300 | 0.926 | 0.923 |
| Word2Vec (DRF) | 300 | 0.787 | 0.785 |
| GloVe + EF (DRF) | 311 | 0.796 | 0.792 |
| Sent2Vec (DRF) | 600 | 0.833 | 0.83 |
| GloVe (DRF) | 300 | 0.824 | 0.822 |
| FastText (FS) | 5 | 0.796 | 0.776 |
| Roberta-Base (FT) | 768 | 0.787 | 0.753 |
| Electra-Base (FT) | 768 | 0.861 | 0.845 |

The system extracts seven features from the text segments: richness of vocabulary (measured by the unique word count), word count, number of stop words, number of coordinating conjunction, number of subordinated conjunction, average word length, and number of interjections. Using CHAT symbols, the system extracts four more features: number of repetitions (using [/]), number of repetitions with reformulations (using [//]), number of errors (using [*]), and number of filler words (using [&]).

In step 20, the system performs deep machine learning on the extracted acoustic and linguistic features, and in step 22, based on the results of the deep learning, the system indicates the likelihood of alzheimer's disease. In these steps, three different settings could be applied: Random Forest with deep pre-trained Features (DRF), fine-tuning of pre-trained models (FT) and training from scratch (FS).

For Deep Random Forest setting, the system extracts features using three pre-trained embeddings: Word2Vec (CBOW) with subword information (pre-trained on Common Crawl), GloVe pre-trained on Common Crawl and Sent2Vec (with uni-grams) pre-trained on English Wikipedia. The procedure is the same for each model: each text segment is represented by the average of the normalised word embeddings. The segment embeddings are then fed to a Random Forest Classifier. In this setting the best performing model is Sent2Vec with unigram representation. Sent2Vec is built on top of Word2Vec, but allows the embedding to incorporate more contextual information (entire sentences) during pre-training.

In the training from scratch setting, models are trained from scratch on the given dataset. The FastText model was found to be fast enough to allow finding of the best hyper-parameters while being a good baseline. With an embedding dimension as low as 5 and with as low as 16 words in its vocabulary, FastText performs competitively compared to most of the Deep Random Forest Settings. Subword information determined by character n-grams are important to the result as explained below.

For the fine tuning setting, pre-trained embeddings (Word2Vec, GloVe, Sent2Vec) or models (Electra, Roberta) are fine-tuned on the data. Electra uses a Generator/Discriminator pre-training technique more efficiently than the Masked Language Modeling approach used by Roberta. Though the results of the two models are approximately the same at the segment level, Electra strongly outperforms Roberta at the participant level. The best models still remain the ones using subword information: GloVe (FT) and Word2Vec (FT). Both of those pre-trained embeddings are fine-tuned with the FastText classifier. The latter turns sentences into character-n-gram augmented sentences (a maximum character n-grams of 6 was found to be optimal). Though FastText from scratch also has the sub-word information, it does not have the pre-trained representation of those sub-words learnt using GloVe or CBOW (Word2Vec).

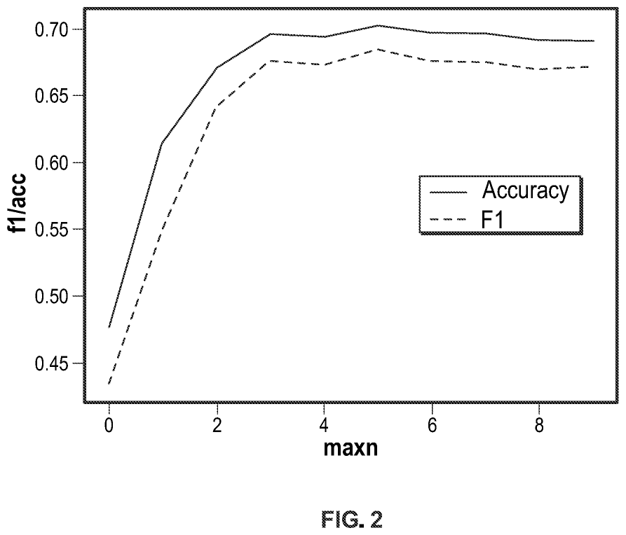

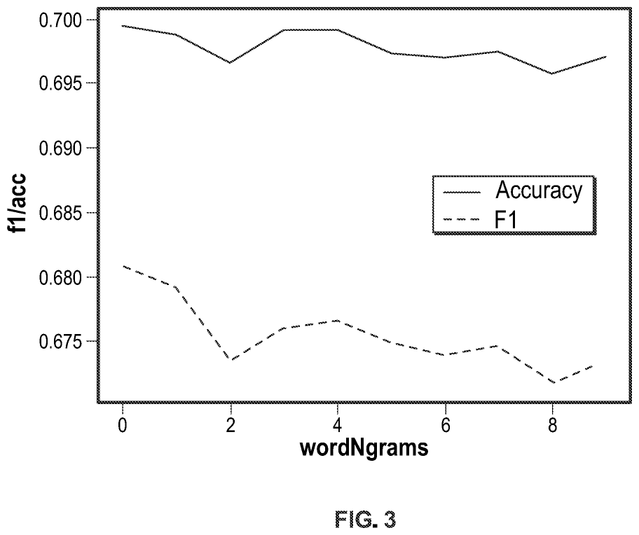

FIGS. 2-3 are charts illustrating testing of the machine learning systems and methods of the present disclosure. Subword information appears to be a key discriminative feature for effective classification. As FIG. 2 shows, not using subword information is detrimental to the discriminative power of the model. As a result, it is possible that in low resource settings like in this case of medical data, taking into account subword information might be the key to good performance. This can be explored further by transforming sentences into phoneme level sentences.

When word order is important, FastText tends to not perform well as it averages the word embeddings of the input sentences without accounting for their original position. This was confirmed by measuring the impact of adding word n-grams as additional features to the classifiers. FIG. 3 shows that adding word n-grams, thus introducing temporality, does not impact the performance or even degrade it.

Though Transformers have subword information through the use of Byte Pair Encoding tokenizer for Roberta and WordPiece tokenizer for Electra, there are too few data points for their large number of parameters.

For the Random Forest Classifier (RF), it was found that the best results on the 6-fold cross validation were obtained using 200 estimators, entropy criterion, square root for the maximum number of features. A StandardScaler (subtracting the mean and scaling to unit variance) was also applied to the features. FastText From Scratch (FS) hyper-parameters are: wordNgrams=1, 100 epochs, max number of character n-grams=6, minimum number of word occurences=100, learning rate of 0.05 and embedding dimension of 5. We kept the same hyper-parameters for FastText fine-tuned except for the dimension that we set to 300 for Word2Vec and GloVe and 600 for Sent2Vec. Roberta-Base and Electra-Base performance was measured on the best hyper-parameters found. The hyper-parameters that were found to work best are: a batch size of 16, 5 epochs, a maximum token length of 128 and a learning rate of 2e-05.

The discriminative importance of the subword information was confirmed by phoneme transcription experiments. We transcribed the segment text into phoneme written pronunciation using CMUDict. The most likely pronunciation is used for words with multiple pronunciations. Thus, “also taking cookies” becomes “ao1 l s ow0 t ey1 k ih0 ng k uh1 k iy0 z.” In several experiments, it always helped to include vowel stress in the pronunciation (0 is no stress, 1 is full stress, 2 is part stress). With stress, there were 66 phones total.

Several text classifiers were trained on the phoneme representation (FastText, Sent2Vec, StarSpace), and FastText was again found to perform best (and fastest). Indeed, our best performance on the final Test set (see Table 6) used only the phoneme representation and FastText classification, along with the audio. However, in 9-fold CV tests with the Train set, the best result was a combination of natural language and phonetic representation (see Table 5).

TABLE 5
Results of 9-fold CV on the Train set for several combined
systems, using simple LR on posterior probability outputs.
Audio represents the LDA posterior probabilities of
Com-ParE2016. Word2Vec and GloVe were text (word-
based) systems and Phonemes are as described above. Age and
speaking rate were added to each system.

| Model | Accuracy |
|---|---|
| GloVe + Phonemes | 0.8981 |
| GloVe + Phonemes + Audio | 0.9074 |
| Word2Vec + Phonemes | 0.9352 |
| Word2Vec + Phonemes + Audio | 0.9352 |

System performance was further tested in the following five system scenarios:

System 1: Audio (LDA posterior probabilities of Com-ParE2016 features)

System 2: Phonemes (as described above)

System 3: Phonemes and Audio

System 4: Phonemes and Word2vec (as described above)
System 5: Phonemes and Audio and Word2Vec

For each combined system (Tables 5 and 6), we appended the age and speaking rate as auxiliary features. Those two variables are well studied for identifying AD.

TABLE 6
Challenge Test Set Results

| Model | Class | Precision | Recall | F1 Score | Accuracy |
|---|---|---|---|---|---|
| System 1 | non-AD | 0.6316 | 0.5 | 0.5581 | 0.6042 |
| | AD | 0.5862 | 0.7083 | 0.6415 | |
| System 2 | non-AD | 0.7407 | 0.8333 | 0.7643 | 0.7708 |
| | AD | 0.8095 | 0.7083 | 0.7656 | |
| System 3 | non-AD | 0.7692 | 0.8333 | 0.8 | 0.7917 |
| | AD | 0.8182 | 0.7500 | 0.7626 | |
| System 4 | non-AD | 0.7308 | 0.7917 | 0.76 | 0.75 |
| | AD | 0.7727 | 0.7083 | 0.7391 | |
| System 5 | non-AD | 0.75 | 0.75 | 0.75 | 0.75 |
| | AD | 0.75 | 0.75 | 0.75 | |

Acoustic features alone are not as discriminative as text features alone. There is indeed a 15 points difference in accuracy between System 1 which mainly uses acoustic features and System 4 which mainly uses text features.

RoBERTa and Electra models performed worse than Word2Vec on this small dataset (see Table 4), and systems 4 and 5 perform worse on the final Test set than just Phonemes alone (see Table 6). However, 9-fold CV on the Train set (see Table 5) found that the best performing system was multiscale (Word2Vec and Phonemes) as well as multimodal (text and audio) (see Table 5). It is believed that this would also give the best result for the Test set if the amount of data were larger.

The effectiveness of using subword features to discriminate between AD and non-AD people can be understood as analogous to data augmentation. Splitting tokens into subwords or mapping them to phonemes reduces the size of the vocabulary and at the same time expands the number of tokens in the training set. Several studies found that AD patients had articulatory difficulties and patterns which would show on the phonetic transcription. Phoneme representations also capture many simple aspects of word-based text models, noting that phoneme 4-grams as used here already include many basic words.

The numbers appended to vowel phonemes are stress indicators according to the convention of CMUdict. Our experiments showed that removing stress always caused a decrease in performance. The discriminative importance of phonetic and articulatory representation in AD patient comes in accordance with previous medical research.

For the phonetic experiments, we used FastText supervised classifier with the following hyperparameters: 4 wordNgrams, an embedding dimension of 20, a learning rate of 0.05, 300 epochs, and a bucket size of 50000. The other hyperparameters were at default. We did not use character n-grams (many phones are already characters).

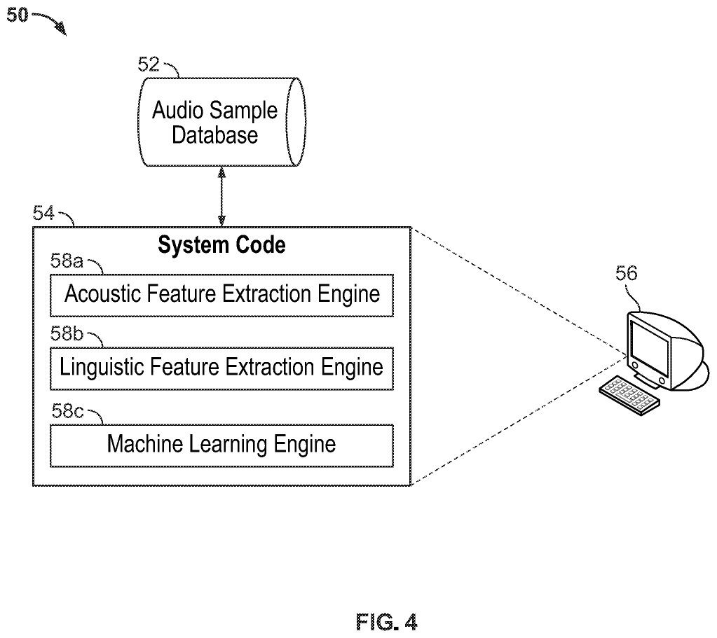

FIG. 4 is a diagram illustrating hardware and software components, indicated generally at 50, capable of being utilized to implement the machine learning systems and methods of the present disclosure. The systems and methods of the present disclosure could be embodied as machine-readable instructions (system code) 54, which could be stored on one or more memories of a computer system and executed by a processor of the computer system, such as computer system 56. Computer system 56 could include a personal computer, a mobile telephone, a server, a cloud computing platform, or any other suitable computing device. The audio samples processed by the code 54 could be stored in an and accessed from an audio sample database 52, which could be stored on the computer system 56 or on some other computer system in communication with the computer system 56. The system code 54 can carry out the processes disclosed herein (including, but not limited to, the processes discussed in connection with FIG. 1), and could include one or more software modules such as an acoustic feature extraction engine 58a (which could extract acoustic features from audio samples as disclosed herein), linguistic feature extraction engine 58b (which could extract linguistic features from the audio samples as disclosed herein), and a machine learning engine 58c (which could perform machine learning on the extracted linguistic and acoustic features to detect Alzheimers' disease, as discussed herein). The system code 54 could be stored on a computer-readable medium and could be coded in any suitable high- or low-level programming language, such as C, C++, C#, Java, Python, or any other suitable programming language.

The machine learning systems and methods disclosed herein provide a multiscale approach to the problem of automatic Alzheimer's Disease (AD) detection. Subword information, and in particular phoneme representation, helps the classifier discriminate between healthy and ill participants. This finding could prove useful in many medical or other settings where lack of data is the norm.

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure. What is desired to be protected by Letters Patent is set forth in the following claims.

What is claimed is:

1. A machine learning system for detecting Alzheimer's disease from one or more audio samples, comprising:
a memory storing one or more audio samples; and
a processor in communication with the memory, the processor programmed to:
retrieve the one or more audio samples from the memory;
process the one or more audio samples to extract acoustic features from audio samples;
process the one or more audio samples to extract linguistic features from the audio samples;
perform machine learning on the extracted acoustic and linguistic features; and
indicate a likelihood of Alzheimer's disease based on output of machine learning performed on the extracted acoustic and linguistic features.

2. The system of claim 1, wherein the processor enhances the one or more audio samples prior to processing the one or more audio samples to extract acoustic features from the audio samples.

3. The system of claim 1, wherein the processor extracts the acoustic features from the one or more audio samples by computing low-level descriptors and statistical functionals of the low-level descriptors.

4. The system of claim 3, wherein the low-level descriptors and statistical functionals of the low-level descriptors are calculated over audio chunks of the one or more audio samples.

5. The system of claim 1, wherein the processor extracts the linguistic features by determining natural language representations from the one or more audio samples.

6. The system of claim 5, wherein the processor extracts the linguistic features by determining phoneme representations from the one or more audio samples.

7. The system of claim 1, wherein the processor performs machine learning on the extracted acoustic and linguistic features using one or more of a Random Forest process with deep pre-trained features, a fine-tuning of pre-trained models, or training from scratch.

8. A machine learning method for detecting Alzheimer's disease from one or more audio samples, comprising the steps of:
processing the one or more audio samples to extract acoustic features from audio samples;
processing the one or more audio samples to extract linguistic features from the audio samples;
performing machine learning on the extracted acoustic and linguistic features; and
indicating a likelihood of Alzheimer's disease based on output of machine learning performed on the extracted acoustic and linguistic features.

9. The method of claim 8, further comprising enhancing the one or more audio samples prior to processing the one or more audio samples to extract acoustic features from the audio samples.

10. The method of claim 8, further comprising extracting the acoustic features from the one or more audio samples by computing low-level descriptors and statistical functionals of the low-level descriptors.

11. The method of claim 10, further comprising calculating the low-level descriptors and statistical functionals of the low-level descriptors over audio chunks of the one or more audio samples.

12. The method of claim 8, further comprising extracting the linguistic features by determining natural language representations from the one or more audio samples.

13. The method of claim 12, further comprising extracting the linguistic features by determining phoneme representations from the one or more audio samples.

14. The method of claim 8, further comprising performing machine learning on the extracted acoustic and linguistic features using one or more of a Random Forest process with deep pre-trained features, a fine-tuning of pre-trained models, or training from scratch.

15. A non-transitory computer-readable medium having computer-readable instructions stored thereon which, when executed by a processor, cause the processor to perform a machine learning method for detecting Alzheimer's disease from one or more audio samples, the instructions comprising:
processing the one or more audio samples to extract acoustic features from audio samples;
processing the one or more audio samples to extract linguistic features from the audio samples;
performing machine learning on the extracted acoustic and linguistic features; and
indicating a likelihood of Alzheimer's disease based on output of machine learning performed on the extracted acoustic and linguistic features.

16. The computer-readable medium of claim 15, wherein the instructions further comprise enhancing the one or more audio samples prior to processing the one or more audio samples to extract acoustic features from the audio samples.

17. The computer-readable medium of claim 15, wherein the instructions further comprise extracting the acoustic features from the one or more audio samples by computing low-level descriptors and statistical functionals of the low-level descriptors.

18. The computer-readable medium of claim 17, wherein the instructions further comprise calculating the low-level descriptors and statistical functionals of the low-level descriptors over audio chunks of the one or more audio samples.

19. The computer-readable medium of claim 15, wherein the instructions further comprise extracting the linguistic features by determining natural language representations from the one or more audio samples.

20. The computer-readable medium of claim 19, wherein the instructions further comprise extracting the linguistic features by determining phoneme representations from the one or more audio samples.

21. The computer-readable medium of claim 15, wherein the instructions further comprise performing machine learning on the extracted acoustic and linguistic features using one or more of a Random Forest process with deep pre-trained features, a fine-tuning of pre-trained models, or training from scratch.
